(19) United States
(12) Patent Application Publication
(10) Pub. No.: US 2015/0272540 A1
(43) Pub. Date: Oct. 1, 2015

(54) MEASUREMENT APPARATUS, MEASUREMENT METHOD, AND ULTRASONIC DIAGNOSIS APPARATUS

(71) Applicant: ADVANTEST CORPORATION, Tokyo (JP)

(72) Inventor: Taiichiro IDA, Gunma (JP)

(21) Appl. No.: 14/621,394

(22) Filed: Feb. 13, 2015

(30) Foreign Application Priority Data
Mar. 25, 2014 (JP) 2014-062182

(51) Int. Cl. A61B 8/08; A61B 8/00; A61B 8/14

(52) U.S. Cl. CPC A61B 8/08; A61B 8/14; A61B 8/5207; A61B 8/463; A61B 8/5223

(57) ABSTRACT

To measure the content percentage of fat in a liver without increasing hardware size, provided is a measurement method and a measurement apparatus including a touching unit that supplies an ultrasonic signal to a body part and receives a response signal that has passed through an inner portion of the body part; a measuring unit that is connected to the touching unit and measures a characteristic of the body part based on the response signal received by the touching unit; and a switching section that switches between connecting the touching unit to the measuring unit and connecting the touching unit to an image generation apparatus that generates an image of a region including the body part in response to the touching unit scanning the region.

The contents of the following Japanese patent application(s) are incorporated herein by reference: No. 2014-062182 filed in JP on Mar. 25, 2014.

### BACKGROUND

1. Technical Field

The present invention relates to a measurement apparatus, a measurement method, and an ultrasonic diagnosis apparatus.

2. Related Art

It is known that the speed of sound passing through physical matter changes according to temperature and that the amount of this change differs according to the type of physical matter. For example, it is known that the speed of sound when propagating through muscle or internal organs, which contain a large amount of water, and the speed of sound propagating through fat cells experience different changes in speed in response to changes in temperature. Therefore, it is known that by applying ultrasonic waves to the liver or the like and measuring the change in speed of a reflected ultrasonic signal in response to a change in temperature of the liver, the liver can be diagnosed as being a fatty liver or not, as shown in International Publication WO 2011/125549, for example.

However, the change in speed of the ultrasonic wave that is dependent on the physical matter in this way is indicated by only a slight difference between approximately +2 m/s·° C. in water and approximately −4 m/s·° C. in fat cells, and therefore the received ultrasonic reflected signal must be processed through an A/D conversion with a high speed sampling rate of at least several hundred MHz. Furthermore, when the measurement point fluctuates due to breathing or the like during measurement, there is an error in the measurement results, and therefore signal processing such as calculating the correlation of a plurality of pieces of data is necessary, which increases the physical dimensions of the measurement apparatus.

### SUMMARY

Therefore, it is an object of an aspect of the innovations herein to provide a measurement apparatus, a measurement method, and an ultrasonic diagnosis apparatus, which are capable of overcoming the above drawbacks accompanying the related art. The above and other objects can be achieved by combinations described in the claims. According to first aspect of the invention, provided is a measurement method and a measurement apparatus including a touching unit that supplies an ultrasonic signal to a body part and receives a response signal that has passed through an inner portion of the body part; a measuring unit that is connected to the touching unit and measures a characteristic of the body part based on the response signal received by the touching unit; and a switching section that switches between connecting the touching unit to the measuring unit and connecting the touching unit to an image generation apparatus that generates an image of a region including the body part in response to the touching unit scanning the region.

According to second aspect of the invention, provided is An ultrasonic diagnosis apparatus including the measurement apparatus according to the first aspect and an image generation apparatus that is connected to the touching unit and generates an image of a region including the body part, in response to the touching unit scanning the region. The ultrasonic diagnosis apparatus detects content percentage of fat at a designated measurement point in the image of the region, by having the switching section connect the touching unit to the measuring unit.

The summary clause does not necessarily describe all necessary features of the embodiments of the present invention. The present invention may also be a sub-combination of the features described above

### BRIEF DESCRIPTION OF THE DRAWINGS

FIG. 1 shows an exemplary configuration of a measurement apparatus 100 according to an embodiment of the present invention, along with an image generation apparatus 300.

FIG. 2 shows a configurational example of the measurement apparatus 100 with the touching unit 10 and the measuring unit 30 connected thereto, according to the present embodiment.

FIG. 3 shows an operational flow of the measurement apparatus 100 according to the present embodiment.

FIG. 4 shows a modification of the measurement apparatus 100 according to the present embodiment.

### DESCRIPTION OF EXEMPLARY EMBODIMENTS

Hereinafter, some embodiments of the present invention will be described. The embodiments do not limit the invention according to the claims, and all the combinations of the features described in the embodiments are not necessarily essential to means provided by aspects of the invention.

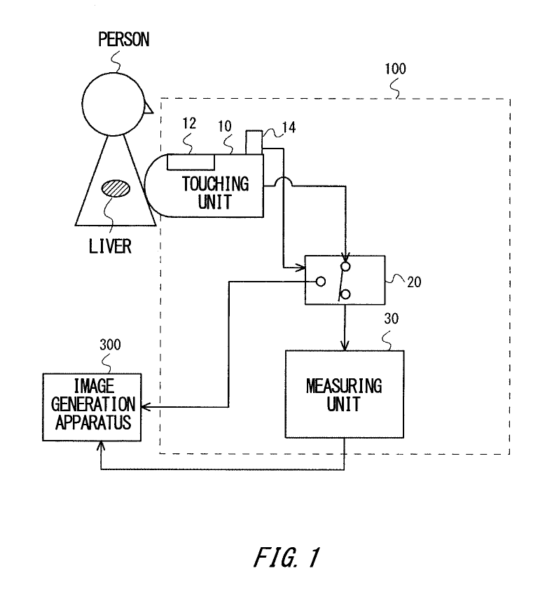

FIG. 1 shows an exemplary configuration of a measurement apparatus 100 according to an embodiment of the present invention, along with an image generation apparatus 300. The measurement apparatus 100 supplies an ultrasonic signal to a body part and receives a response signal that has passed through an inner portion of this body part. The measurement apparatus 100 is connected to the image generation apparatus 300, and switches between a function of supplying the response signal to the image generation apparatus 300 to generate an image of a region including the body part and a function of measuring characteristics or the like of the body part according to the response signal. The present embodiment describes an example in which the measurement apparatus 100 detects physical matter contained in the body part according to the response signal.

In a case where the measurement apparatus 100 supplies the response signal to the image generation apparatus 300, the measurement apparatus 100 and the image generation apparatus 300 form an ultrasonic diagnosis apparatus or ultrasonic measurement apparatus that generates an image of a region containing a body part of an animal, particularly a human. In other words, the image generation apparatus 300 may be a portion of an ultrasonic measurement apparatus or an ultrasonic diagnosis apparatus, for example. In this case, the measurement apparatus 100 is connected to the image generation apparatus 300 instead of a probe that handles the ultrasonic signal of the image generation apparatus 300. The measurement apparatus 100 includes a touching unit 10, a switching section 20, and a measuring unit 30.

The touching unit 10 touches a body, supplies an ultrasonic signal to the body part, and receives a response signal that has passed through the inner portion of the body part. The touching unit 10 faces a measurement point or measurement region of the inner portion of the body, touches a location near this measurement point or the like, and supplies the ultrasonic signal, for example, in a manner to efficiently irradiate the measurement point or the like with the ultrasonic signal. Furthermore, the touching unit 10 receives, as a response signal, a reflected signal that has been reflected from the measurement point or measurement region. The touching unit 10 preferably has a size, shape, and weight that enable the touching unit 10 to be held in the hand of a user operating the measurement apparatus 100, i.e. the person performing the measurement, and is used to touch the body of the person undergoing the measurement. The touching unit 10 includes a heating section 12 and a switching button 14.

The heating section 12 heats the inner portion of the body part. The heating section 12 heats the inner portion of the body part to be a predetermined temperature. The heating section 12 is provided within the touching unit 10, and is formed integrally with the touching unit 10. Instead, the heating section 12 may be provided outside of the touching unit 10.

The heating section 12 includes an irradiating section that radiates ultrasonic waves or electromagnetic waves, for example, and increases the temperature within the body. The heating section 12 may include a plurality of irradiating sections, and may perform irradiation by focusing ultrasonic waves or electromagnetic waves at a predetermined location within the body. Instead of or in addition to the heating section 12, the touching unit 10 may include a cooling section that cools the inside of the body. The heating section 12 may touch the body to supply the ultrasonic waves or the like.

In a case where the measurement apparatus 100 measures characteristics or the like of the body part according to the response signal, the heating section 12 stops radiating the ultrasonic waves or electromagnetic waves after the temperature within the body has risen, for example. Then, the measurement apparatus 100 supplies the ultrasonic signal until the raised temperature within the body returns to the original temperature, and receives a response signal. The heating section 12 may stop operating when the measurement apparatus 100 supplies the response signal to the image generation apparatus 300. Instead, the heating section 12 may maintain the temperature within the body at a predetermined temperature or designated temperature.

The switching button 14 supplies the switching section 20 with the control signal for controlling the switching section 20, in response to input from the user, for example. The switching button 14 is provided in the touching unit 10 at a location enabling manipulation of the switching button 14 in a state where the user holds the touching unit 10 in a hand. In other words, the user manipulates the switching button 14 while touching the body with the touching unit 10, and can control the switching between the measurement by the measurement apparatus 100 and the image generation by the image generation apparatus 300. Furthermore, in addition to the switching button 14, the touching unit 10 may further include ON and OFF buttons for the heating by the heating section and/or generation of the ultrasonic signal.

The switching section 20 switches between a state in which the touching unit 10 is connected to the measuring unit 30 and a state in which the touching unit 10 is connected to the image generation apparatus 300 that generates an image of a region including the body part in response to this region being scanned by the touching unit. The switching section 20 receives the control signal from the switching button 14 and, according to the input to the switching button 14, switches whether the touching unit 10 is connected to the measuring unit 30 or to the image generation apparatus 300.

In a case where the image generation apparatus 300 includes an input connector, for example, the switching section 20 includes a connector that engages with this input connector, and is connected to the image generation apparatus 300. Furthermore, the switching section 20 may be connected to an internal wire of the image generation apparatus 300. The switching section 20 may include one or more one-input/two-output switches, for example.

The switching section 20 may be formed integrally with the measuring unit 30. As another example, the switching section 20 may be formed integrally with the touching unit 10. In this case, the switching section 20 and the image generation apparatus 300 may be connected by a cable or the like. As yet another example, the touching unit 10, the switching section 20, and the measuring unit 30 may be formed integrally.

The measuring unit 30 is connected to the touching unit 10 and receives the response signal that is received in response to the ultrasonic signal supplied by the touching unit 10. The measuring unit 30 measures the characteristics of the body part based on the response signal received by the touching unit 10. For example, the measuring unit 30 detects the physical matter contained in the body part. The measuring unit 30 preferably has organs, internal organs, tissue, or the like that contain homogenous physical matter as the body part to be a measurement target. The present embodiment describes an example in which the measuring unit 30 uses a liver as the body part to be the measurement target.

The measuring unit 30 may transmit the measurement results to the image generation apparatus 300 and display the measurement results in a display section of the image generation apparatus. In this case, the measuring unit 30 is connected to the image generation apparatus 300 and supplies the image generation apparatus 300 with the measurement result data. Furthermore, the switching section 20 connects the touching unit 10 to the measuring unit 30, and the measuring unit 30 transmits to the image generation apparatus 300 a timing signal for displaying the measurement results in the display section of the image generation apparatus 300, in response to the measurement results being within a predetermined range of values. Instead of or in addition to this, the measuring unit 30 may include a display section that displays the measurement results.

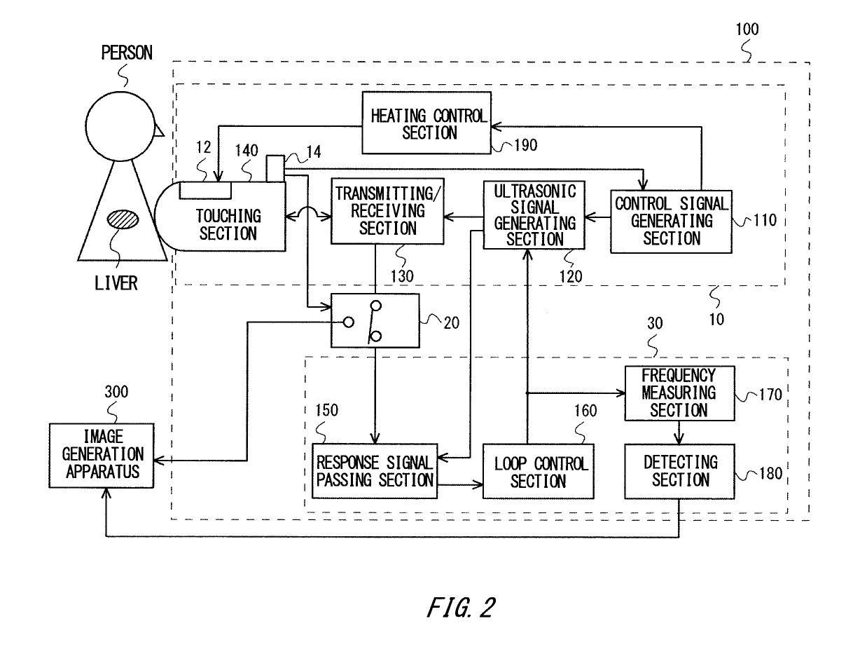

The measuring unit 30 measures the characteristics of the liver according to phase change of the response signal caused by temperature change in an inner portion of the liver. FIG. 2 is used to describe an example in which the measurement apparatus 100 measures the characteristics of the liver of a person using the measuring unit 30.

FIG. 2 shows a configurational example of the measurement apparatus 100 with the touching unit 10 and the measuring unit 30 connected thereto, according to the present embodiment. The measurement apparatus 100 irradiates the liver of the person with an ultrasonic signal, and receives the ultrasonic signal that has passed through the liver as a response signal. The measurement apparatus 100 receives a response signal that is a reflected signal that has been reflected from the inner portion of the liver, for example. FIG. 2 shows an example in which the touching unit 10 supplies the liver with an ultrasonic signal and receives a reflected signal generated at a border between lobes of the liver, e.g. the right lobe, the left lobe, the caudate lobe, and the quadrate lobe, as the response signal, and the measuring unit 30 measures the content percentage of fat in the liver.

The measurement apparatus 100 includes a control signal generating section 110, an ultrasonic signal generating section 120, a transmitting/receiving section 130, a touching section 140, a response signal passing section 150, a loop control section 160, a frequency measuring section 170, a detecting section 180, and a heating control section 190. Specifically, the touching unit 10 includes the control signal generating section 110, the ultrasonic signal generating section 120, the transmitting/receiving section 130, the touching section 140, and the heating control section 190, while the measuring unit 30 includes the response signal passing section 150, the loop control section 160, the frequency measuring section 170, and the detecting section 180.

The control signal generating section 110 generates a control signal for generating the ultrasonic signal, and supplies this control signal to the ultrasonic signal generating section 120. The control signal generating section 110 may generate the control signal for generating the ultrasonic signal, in response to the switching button 14 being turned ON. Furthermore, in a case where the touching unit 10 includes an ON and OFF button for generating the ultrasonic signal, the control signal generating section 110 may generate the control signal for generating the ultrasonic signal in response to this button being turned ON. Instead, the control signal generating section 110 may constantly output the ultrasonic signal when the power source of the measurement apparatus 100 is ON.

The control signal generating section 110 is connected to the heating control section 190, and may supply a control signal for changing the temperature of the inner portion of the liver to the heating control section 190. In this case, for example, the control signal generating section 110 supplies the heating control section 190 with the control signal for changing the temperature of the inner portion of the liver and then, after a predetermined time has passed, supplies the ultrasonic signal generating section 120 with the control signal for generating the ultrasonic signal.

The ultrasonic signal generating section 120 is connected to the control signal generating section 110, and generates the ultrasonic signal according to the control signal received from the control signal generating section 110. The ultrasonic signal generating section 120 supplies the ultrasonic signal to the portion including the liver, through the transmitting/receiving section 130 and the touching section 140. The ultrasonic signal generating section 120 includes a puller for generating ultrasonic waves and outputs a high-voltage pulse in which the wave peak reaches from tens to a hundred and tens of volts, for example.

The transmitting/receiving section 130 is connected to the ultrasonic signal generating section 120 and the touching section 140, and supplies the touching section 140 with the ultrasonic signal received from the ultrasonic signal generating section 120. When the connection of the switching section 20 is set to the measuring unit 30, the transmitting/receiving section 130 is connected to the signal passing section 150 via the switching section 20, and supplies the signal passing section 150 with the response signal received form the touching section 140. When the connection of the switching section 20 is set to the image generation apparatus 300, the transmitting/receiving section 130 supplies the image generation apparatus 300 with the response signal via the switching section 20. The transmitting/receiving section 130 includes a transmission/reception switch for the ultrasonic signal, for example.

The touching section 140 touches the body and receives the ultrasonic signal. The touching section 140 may include the heating section 12. In this case, the heating section 12 may touch the body in the same manner as the touching section 140, and may supply the ultrasonic signal to the measurement point or the like.

The response signal passing section 150 is provided between the ultrasonic signal generating section 120 and the loop control section 160, passes the response signal supplied from the touching section 140 to the loop control section 160, and attenuates the ultrasonic signal that leaks from the transmitting/receiving section 130. The response signal passing section 150 includes an amplification circuit with variable amplification and/or a switch circuit that amplify or attenuate a signal input thereto, according to a timing signal input thereto.

The response signal passing section 150 is connected to the ultrasonic signal generating section 120 and acquires the timing at which the ultrasonic signal is attenuated from the ultrasonic signal generating section 120, for example. Instead, the response signal passing section 150 may be connected to the control signal generating section 110 and may acquire the timing at which the ultrasonic signal is attenuated from the control signal generating section 110.

The loop control section 160 is connected to the ultrasonic signal generating section 120 and the response signal passing section 150, and supplies the ultrasonic signal generating section 120 with the control signal for generating the ultrasonic signal, in response to receiving the response signal from the liver from the response signal passing section 150. Specifically, the ultrasonic signal generating section 120, the transmitting/receiving section 130, the touching section 140, the liver, the response signal passing section 150, and the loop control section 160 form a loop path through which a signal based on the ultrasonic signal is sequentially transmitted.

The frequency measuring section 170 is connected to the loop control section 160 and measures the repeating frequency of the control signal that is repeatedly supplied to the ultrasonic signal generating section 120 by the loop control section 160 according to the response signal. The frequency measuring section 170 may be a frequency counter that counts how many times the electrical signal is input per unit time, to measure the frequency of this electrical signal. The frequency measuring section 170 supplies the detecting section 180 with the measured frequency.

The detecting section 180 is connected to the frequency measuring section 170 and detects the physical material contained in the liver through which the ultrasonic signal has passed, based on the frequency measured by the frequency measuring section 170. For example, the detecting section 180 detects a change in the frequency measured by the frequency measuring section 170, and detects the content percentage of fat in the liver.

The heating control section 190 controls the heating of the heating section 12. The heating control section 190 controls the heating of the heating section 12 in response to receiving the control signal from the control signal generating section 110. As another example, the heating control section 190 may control the heating of the heating section 12 in response to the switching section 20 connecting the touching unit 10 to the measuring unit 30. In this case, the control signal generating section 110 may supply the heating control section 190 with the control signal for changing temperature in response to receiving the control signal from the switching button 14.

The above describes an example in which, in the measurement apparatus 100 of the present embodiment, the switching section 20 is connected between the transmitting/receiving section 130 and the response signal passing section 150. Instead, the switching section 20 may be connected between the response signal passing section 150 and the loop control section 160.

The measurement apparatus 100 of the present embodiment described above forms a loop path through which the ultrasonic signal is supplied to the liver according to the received response signal, acquires speed information for the response signal by measuring the signal frequency at which the signal travels around the loop path, and detects the physical matter contained in the liver based on the speed change of the response signal. The operation of the measurement apparatus 100 is described below using an operational flow.

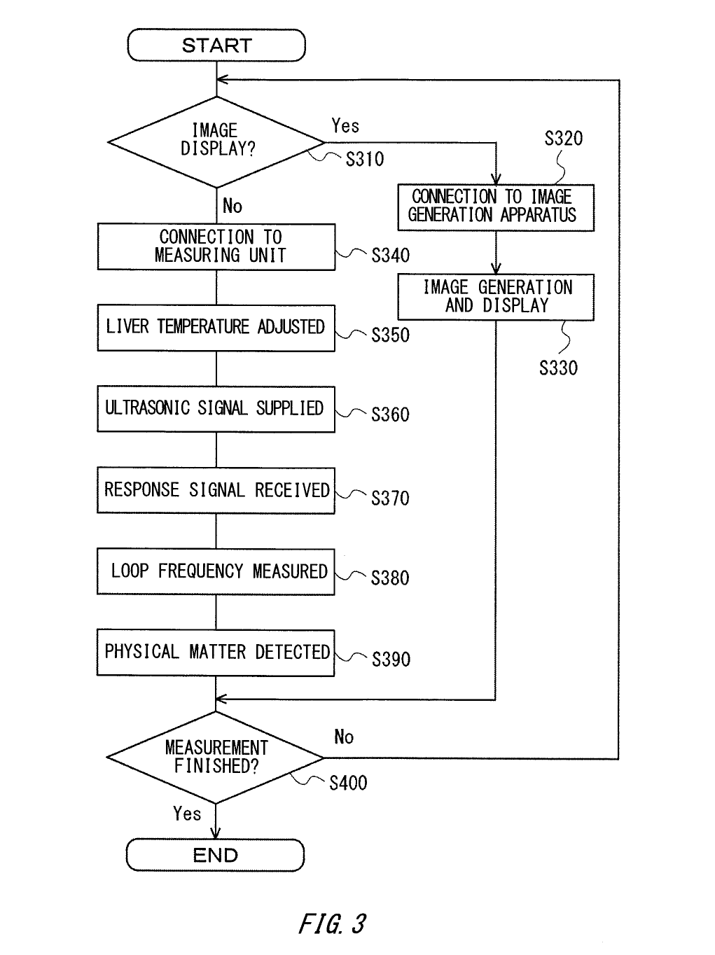

FIG. 3 shows an operational flow of the measurement apparatus 100 according to the present embodiment. The following describes an example in which the measurement apparatus 100 of the present embodiment displays an image of the liver and detects the content percentage of fat by performing steps S310 to S400 shown in FIG. 3.

First, the measurement apparatus 100 switches whether the touching unit 10 is connected to the measuring unit 30 or to the image generation apparatus 300, according to the state of the switching button 14. In a case were the initial setting or input from the user indicates that the state of the switching button 14 is to be image display (S310: Yes), the switching section 20 connects the touching unit 10 to the image generation apparatus 300 (S320).

The ultrasonic signal generating section 120 supplies the ultrasonic signal to the liver of the person. For example, the control signal generating section 110 supplies the ultrasonic signal generating section 120 with the control signal for generating the ultrasonic signal, in response to the user manipulating the button for turning ON or OFF the generation of the ultrasonic signal to be ON.

The ultrasonic signal is supplied into the body from the touching section 140 and the image generation apparatus 300 receives, via the switching section 20, the response signal output in response to this ultrasonic signal. The image generation apparatus 300 generates an image of the inside of the person to which the ultrasonic signal was supplied, according to the response signal, and outputs this image to the display section (S330). Here, in order to generate the image of the inside of the body of the person, the touching unit 10 may scan a region that is to be observed while touching the body.

In other words, when the touching unit 10 is connected to the image generation apparatus 300, the image generation apparatus 300 generates an image of a region including the liver in response to the touching unit 10 scanning this region. In a case where the ultrasonic signal supplied by the touching unit 10 is to generate an image of a region including the liver, the radiation direction of the ultrasonic signal may be adjusted or scanned toward this region, and in this case the touching unit 10 may be fixed while touching the body.

When the measurement is not yet finished (S400: No) and there has been no change in the input of the switching button 14 (S310: Yes), the measurement apparatus 100 keeps the connection of the switching section 20 set to the image generation apparatus 300 (S320) and continues the image generation (S330). When the input of the user or the initial settings indicate that the state of the switching button 14 is to detect physical matter (S310: No), the measurement apparatus 100 connects the touching unit 10 to the measuring unit 30 (S340).

The control signal generating section 110 supplies the heating control section 190 with the control signal for changing the temperature of the inner portion of the liver, to adjust the temperature of the inner portion of the liver before the liver is supplied with the ultrasonic signal from the ultrasonic signal generating section 120 (S350). For example, the control signal generating section 110 transmits the control signal for changing the temperature to the heating control section 190 in response to receiving a control signal from the switching button 14. In this way, prior to supplying the ultrasonic signal, the control signal generating section 110 supplies a control signal for increasing the temperature of the inner portion of the liver to a predetermined temperature. The heating control section 190 controls the heating section 12 to adjust the temperature of the inner portion of the liver to be the predetermined temperature, according to this control signal.

Next, the control signal generating section 110 supplies the ultrasonic signal generating section 120 with the control signal for generating the ultrasonic signal, and the ultrasonic signal is supplied to the inner portion of the liver (S360). The control signal generating section 110 raises the temperature of the inner portion of the liver to the predetermined temperature, and then supplies the ultrasonic signal generating section 120 with the control signal for generating the ultrasonic signal.

For example, the control signal generating section 110 supplies the control signal to the heating control section 190 and then, after a predetermined time has passed, supplies the ultrasonic signal generating section 120 with the control signal for generating the ultrasonic signal. In this case, the actual transition time needed for the temperature of the inner portion of the liver to change from when the heating control section 190 begins adjusting the temperature within the liver is measured in advance, and the time at which the ultrasonic signal generating section 120 supplies the control signal may be determined in advance according to the transition time measurement result. In this way, the measurement apparatus 100 can measure the temperature conditions from when the temperature of the liver is raised to the predetermined temperature to when the temperature of the liver has dropped back down to normal body temperature.

The ultrasonic signal generating section 120 supplies the ultrasonic signal to the inner portion of the liver through the transmitting/receiving section 130 and the touching section 140, according to the received control signal. Here, the ultrasonic signal generating section 120 may notify the response signal passing section 150 concerning the timing at which the ultrasonic signal is generated. The response signal passing section 150 acquires the timing at which the ultrasonic signal received from the ultrasonic signal generating section 120 is generated, as the timing at which the input signal is attenuated, and may attenuate the input signal from when this timing is acquired until a predetermined time has passed.

If there is an amplification circuit included, for example, the response signal passing section 150 decreases the amplification amount of the amplification circuit to attenuate the signal strength of the signal component supplied to the loop control section 160. Furthermore, if there is a switch circuit included, the response signal passing section 150 switches this switch circuit to cut off the electrical connection between the transmitting/receiving section 130 and the loop control section 160. In this way, the response signal passing section 150 attenuates the input signal in synchronization with the generation timing of the ultrasonic signal, even when a portion of the ultrasonic signal generated by the ultrasonic signal generating section 120 is leaked from the transmitting/receiving section 130 and input, and therefore the response signal passing section 150 can reduce the noise component and prevent incorrect operation of the loop control section 160.

Here, the response signal passing section 150 may determine in advance the time during which the input signal continues to be attenuated, according to the time during which the pulse is continuously generated as the ultrasonic signal by the ultrasonic signal generating section 120, and the attenuation of the input signal may be stopped after this predetermined time has passed. In this way, every time the ultrasonic signal generating section 120 generates the ultrasonic signal, the response signal passing section 150 can repeatedly switch whether the input signal is being attenuated, thereby reducing or blocking out the signal components other than the response signal.

Next, the touching section 140 receives the response signal, which is the reflected signal of the ultrasonic signal reflected from the inner portion of the liver (S370). The touching section 140 supplies the response signal passing section 150 with the received response signal through the transmitting/receiving section 130 and the switching section 20. The response signal passing section 150 passes the received response signal to the loop control section 160. Specifically, the response signal passing section 150 sets the amplification amount of the amplification circuit and/or the switches the switch circuit such that the response signal is passed to the loop control section 160.

For example, during periods other than the period in which a portion of the ultrasonic signal generated by the ultrasonic signal generating section 120 is leaked from the transmitting/receiving section 130 and input, the response signal passing section 150 passes the signal input thereto to the loop control section 160. Furthermore, the response signal passing section 150 may determine the timing at which the response signal is passed to the loop control section 160 according to the distance between the inner portion of the liver and the touching section 140.

The response signal to be passed to the loop control section 160 is the reflected signal that has been reflected from the inner portion of the liver, and therefore the timing at which the response signal is input to the response signal passing section 150 is determined according to the distance between the touching section 140 and the border between lobes in the inner portion of the liver, for example. Accordingly, the passage time corresponding to the distance that can be obtained between the touching section 140 and the inner portion of the liver is preferably obtained in advance based on information such as the position of the liver of the person and the position where the touching section 140 touches the person. The response signal passing section 150 may perform the setting of the amplification amount of the amplification circuit and/or the switching of the switch such that the response signal is passed during the passage time.

Instead of or in addition to the above, the response signal passing section 150 may pass a signal that exceeds a predetermined amplitude value and may attenuate a signal that is below this amplitude value. In this way, the loop control section 160 can reduce or cut off signals that have an amplitude value below the amplitude value that can be predicted for the response signal. Furthermore, when the amplitude value of a signal leaked from the transmitting/receiving section 130 and input, for example, is less than the predetermined amplitude value, the response signal passing section 150 need not perform the process to attenuate the input signal. If the response signal passing section 150 includes an amplification circuit, the response signal passing section 150 may amplify the signal to be passed, and then pass the resulting signal.

Next, upon receiving the response signal passed by the response signal passing section 150, the loop control section 160 supplies the control signal for generating the ultrasonic signal to the ultrasonic signal generating section 120. The ultrasonic signal generating section 120 generates the ultrasonic signal according to this control signal, and supplies the ultrasonic signal to the inner portion of the liver.

In the manner described above, the measurement apparatus 100 of the present embodiment supplies the ultrasonic signal to the liver in response to receiving the response signal that has passed through the liver, and therefore the supply of this ultrasonic signal and the reception of this response signal are repeated. Specifically, a signal based on the ultrasonic signal travels around the loop path formed by the ultrasonic signal generating section 120, the transmitting/receiving section 130, the touching section 140, the liver, the touching section 140, the switching section 20, the response signal passing section 150, and the loop control section 160.

The frequency measuring section 170 measures the loop frequency at which the signal based on the ultrasonic signal travels around the loop path (S380). Specifically, the frequency measuring section 170 measures the speed of the signal passing through the loop path and the loop frequency determined according to the loop length, and therefore detects the frequency corresponding to the response speed. In other words, the speed information of the signal passing through the loop path can be acquired according to the frequency measured by the frequency measuring section 170. For example, when there is a difference in the speed at which the ultrasonic signal passes through different physical matter, the frequency measuring section 170 can detect the difference between these types of physical matter as the difference in the loop frequency.

As an example, the water, fat, and the like contained in the liver has an almost uniform distribution in the inner portion of the liver, and therefore the speed of the ultrasonic wave passing through the liver depends on the amount of water, fat, and the like contained in the liver. Accordingly, a liver in which the content percentage of water is low enough and the content percentage of fat is high enough to create a difference in speed when compared to a normal liver can be detected due to the difference in the loop frequency.

Furthermore, even when the content percentage of the physical material or between physical materials is such that it is difficult to detect this difference as the difference in the loop frequency, there are cases where this difference can still be detected by utilizing the temperature dependent nature of the loop frequency. For example, the temperature dependency of the speed of an ultrasonic wave is approximately +2 m/s·° C. in water and approximately −4 m/s·° C. in fat cells, thereby exhibiting different directions of change in the speed relative to temperature. Accordingly, in the case of a liver containing a greater amount of water, the loop frequency decreases when the temperature decreases, while in the case of a liver containing a greater amount of fat, the loop frequency increases when the temperature decreases, and therefore the frequency measuring section 170 outputs the increase or decrease in the frequency corresponding to the content percentage of such physical matter.

The detecting section 180 detects the physical matter contained in the liver based on the change in frequency corresponding to the change in temperature of the inner portion of the liver (S390). Furthermore, the detecting section 180 may detect the content percentage of fat in the liver based on the slope of the change in frequency corresponding to the temperature change. For example, when the temperature dependency has a positive sign when the ultrasonic wave speed decreases, in response to there being a decrease in the loop frequency when the temperature decreases, the detecting section 180 detects that the content percentage of water in the liver is high. Here, by measuring in advance the speed change corresponding to the content percentage of fat in the liver and comparing this to the measurement results, the detecting section 180 may identify the content percentage of fat in the liver of the measurement target.

In the manner described above, the measurement apparatus 100 of the present embodiment acquires speed information for the ultrasonic signal passing through the inner portion of the liver by measuring the frequency of the signal travelling around the loop path. Accordingly, the measurement apparatus 100 can detect the speed of the response signal and changes in the speed without performing an A/D conversion with a high-speed sampling rate.

Furthermore, the frequency measuring section 170 can measure the loop frequency using a frequency counter. The frequency counter can improve the accuracy of the measured frequency by using a liquid crystal oscillator or the like to improve the time accuracy, and therefore the accuracy of the detected speed can be increased regardless of the sampling rate.

The frequency counter can easily perform measurement for a high frequency that exceeds 100 MHz, for example, by using a prescaler (frequency divider) to divide the input frequency and then measuring the frequency. Accordingly, the measurement apparatus 100 can improve the design freedom of the loop length. In this way, the measurement apparatus 100 can accurately measure the change in speed of the response signal, and can more accurately detect the content percentage of water or fat contained in the liver of the measurement target.

Furthermore, the frequency counter counts the pulses input per unit time, and can therefore finish the frequency measurement within this unit of time. Accordingly, even when a person is breathing, the frequency measuring section 170 can perform the frequency measurement faster than the person breaths, and can therefore reduce the effect of breathing on the measurement results. Furthermore, the frequency measuring section 170 can perform more frequency measurements during a period when the temperature of the liver drops back to normal body temperature after being increased to a predetermined temperature, and can therefore measure the change in frequency relative to change in temperature with a higher (temperature) resolution.

Yet further, by periodically measuring the liver of the person being targeted, the measurement apparatus 100 can detect the fat content percentage of the liver increasing or decreasing in response to the lifestyle of the person being measured, for example. The detecting section 180 may also have a simple diagnosis function to identify a liver as a fatty liver when the fat content of the liver is greater than or equal to a predetermined value. In the manner described above, the measurement apparatus 100 of the present embodiment can easily detect the physical matter contained in a liver without increasing the size of the apparatus by using high-speed digital signal processing.

The detecting section 180 may supply the image generation apparatus 300 with the detection results, and in this case, the image generation apparatus 300 outputs the detection results to the display section. The image generation apparatus 300 may display the detection results along with the image generated before the connection to the measuring unit 30 is made. Instead of or in addition to this, the measuring unit 30 may include a display section to display the measurement results and the detecting section 180 may display the detection results in the display section of the measuring unit 30.

The detecting section 180 supplies the detection results to the image generation apparatus 300, in response to the detection results being within a predetermined range, for example. In this way, when a measurement error occurs, e.g. when the touching section 140 does not achieve sufficient contact with the body, an abnormal value can be prevented from being displayed as the measurement value in the display section. Furthermore, in this way, a warning lamp or the like may be provided in the touching unit 10 to provide notification through light to the user manipulating the touching unit 10 when it is determined that a measurement error has occurred.

When input indicating the end of measurement is made, e.g. when the power supply of the apparatus is turned OFF, the measurement apparatus 100 ends the measurement (S400: Yes). Furthermore, when measurement continues (S400: No), the measurement apparatus 100 returns to step S310 and switches the switching section 20 according to the state of the switching button 14.

In this way, the measurement apparatus 100 can switch between image generation by the image generation apparatus 300 and physical matter detection by the measurement apparatus 100. Therefore, the user can confirm the image with the image generation apparatus 300 and then begin detection of the physical matter at the measurement point or the like. In this case, the image generation apparatus 300 may generate an image of the region including the liver and then, by having the switching section 20 connect the touching unit 10 to the measuring unit 30, the measuring unit 30 may detect the content percentage of fat at the designated measurement point in the image of this region.

Furthermore, the measurement apparatus 100 checks the image from the image generation apparatus 300 and then changes the temperature of the measurement region or the like, and therefore heating can be started after confirming that the measurement region does not include locations where heating should be prevented such as arteries, for example, thereby enabling safe measurement. Yet further, the measurement apparatus 100 is connected to an existing ultrasonic measurement apparatus or ultrasonic diagnosis apparatus that generates an image, and has functions other than image detection, and therefore these functions can be added to existing apparatuses with reduced cost.

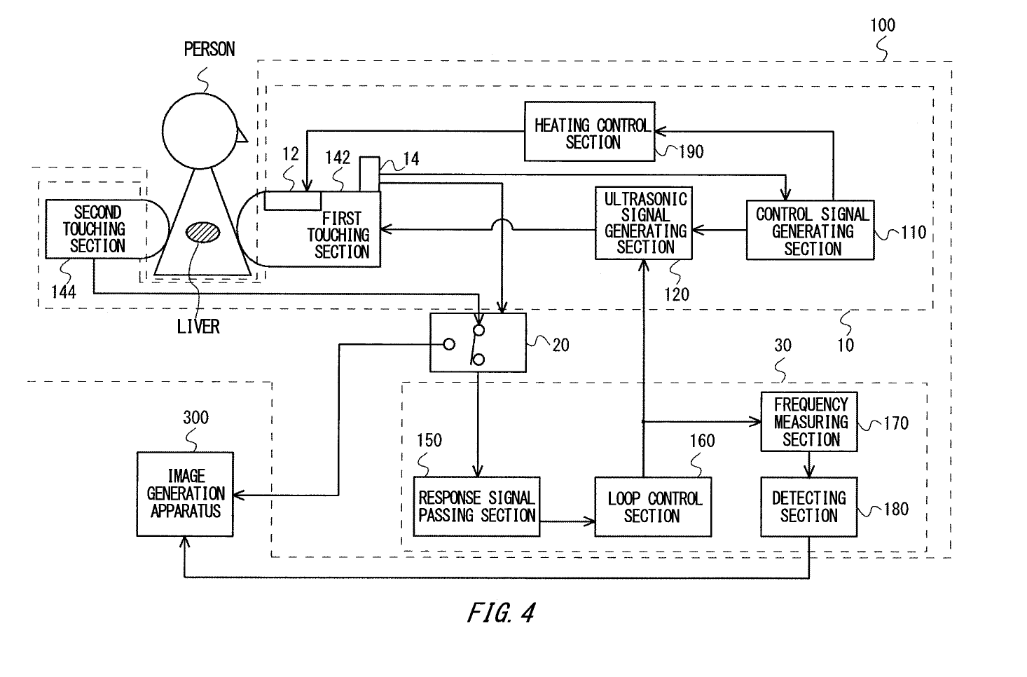

FIG. 4 shows a modification of the measurement apparatus 100 according to the present embodiment. In the measurement apparatus 100 of the present modification, components having substantially the same operation as components in the measurement apparatus 100 according to the embodiment shown in FIG. 2 are given the same reference numerals, and redundant descriptions are omitted. The measurement apparatus 100 of the present modification includes a first touching section 142 and a second touching section 144.

The first touching section 142 touches a human body and supplies the liver of the inner portion of the person with an ultrasonic signal generated by the ultrasonic signal generating section 120. In other words, the first touching section 142 includes at least the function of supplying the liver of the inner portion of the person with an ultrasonic signal, which is a function of the touching section 140 according to the embodiment shown in FIG. 2. Furthermore, the first touching section 142 includes a heating section 12, in the same manner as the touching section 140.

The second touching section 144 touches the human body and receives the ultrasonic signal that has passed through the inner portion of the liver as a response signal. In other words, the second touching section 144 includes at least the function of receiving the response signal, which is a function of the touching section 140 according to the embodiment shown in FIG. 2.

When the switching section 20 connects the second touching section 144 to the measuring unit 30, the second touching section 144 supplies the loop control section 160 with the response signal, through the response signal passing section 150 and the switching section 20. When the switching section 20 connects the second touching section 144 to the image generation apparatus 300, the second touching section 144 supplies the image generation apparatus 300 with the response signal through the switching section 20.

The loop control section 160 causes the ultrasonic signal to be generated from the ultrasonic signal generating section 120 according to the response signal, and supplies the generated ultrasonic signal to the first touching section 142. In this way, the measurement apparatus 100 of the present modification creates a loop path formed by the ultrasonic signal generating section 120, the transmitting/receiving section 130, the first touching section 142, the liver, the second touching section 144, the switching section 20, the response signal passing section 150, and the loop control section 160.

As a result, the second touching section 144 can receive the response signal based on the ultrasonic signal supplied from the first touching section 142 to the liver, from a location differing from the location where the first touching section 142 touches the human body. Therefore, the touching sections can be designed as separate and independent components on the transmission side and the reception side, thereby increasing the design freedom. Furthermore, a portion of the ultrasonic signal supplied from the first touching section 142 on the transmission side is prevented from leaking to the second touching section 144 on the reception side. Yet further, by optimizing the location at which each touching section touches the body, an ultrasonic signal that has passed through organs and tissue from the first touching section 142 can be received by the second touching section 144.

In the manner described above, the measurement apparatus 100 of the present modification divides the touching section 140 that touches the body into the first touching section 142 on the transmission side and the second touching section 144 on the reception side, to transmit and receive the ultrasonic signal. In this case as well, the measurement apparatus 100 can cause the signal based on the ultrasonic signal to travel around the loop path, acquire the speed information indicating the speed at which the ultrasonic signal travels through the liver or the like, and detect the physical matter included in the liver.

While the embodiments of the present invention have been described, the technical scope of the invention is not limited to the above described embodiments. It is apparent to persons skilled in the art that various alterations and improvements can be added to the above-described embodiments. It is also apparent from the scope of the claims that the embodiments added with such alterations or improvements can be included in the technical scope of the invention.

The operations, procedures, steps, and stages of each process performed by an apparatus, system, program, and method shown in the claims, embodiments, or diagrams can be performed in any order as long as the order is not indicated by “prior to,” “before,” or the like and as long as the output from a previous process is not used in a later process. Even if the process flow is described using phrases such as “first” or “next” in the claims, embodiments, or diagrams, it does not necessarily mean that the process must be performed in this order.

What is claimed is:

1. A measurement apparatus comprising:
a touching unit that supplies an ultrasonic signal to a body part and receives a response signal that has passed through an inner portion of the body part;
a measuring unit that is connected to the touching unit and measures a characteristic of the body part based on the response signal received by the touching unit; and
a switching section that switches between connecting the touching unit to the measuring unit and connecting the touching unit to an image generation apparatus that generates an image of a region including the body part in response to the touching unit scanning the region.

2. The measurement apparatus according to claim 1, wherein
the touching unit includes a switching button, and
the switching section switches between connecting the touching unit to the measuring unit and connecting the touching unit to the image generation apparatus in response to input to the switching button.

3. The measurement apparatus according to claim 1, wherein
the touching unit includes a heating section that heats the inner portion of the body part.

4. The measurement apparatus according to claim 3, wherein
the heating section heats the inner portion of the body part to a predetermined temperature.

5. The measurement apparatus according to claim 3, wherein
the touching unit includes a heating control section that controls the heating of the heating section.

6. The measurement apparatus according to claim 5, wherein
the heating control section controls the heating of the heating section in response to the switching section connecting the touching unit to the measuring unit.

7. The measurement apparatus according to claim 4, wherein
the measuring unit measures the characteristic of the body part according to a phase change of the response signal that accompanies a temperature change of the inner portion of the body part.

8. The measurement apparatus according to claim 1, wherein
the switching section is provided integrally with the measuring unit.

9. The measurement apparatus according to claim 1, wherein
the switching section is provided integrally with the touching unit.

10. The measurement apparatus according to claim 1, wherein
the measuring unit transmits a measurement result to the image generation apparatus and causes the measurement result to be displayed in a display section of the image generation apparatus.

11. The measurement apparatus according to claim 10, wherein
in response to the switching section connecting the touching unit to the measuring unit and the measurement result being within a predetermined range of values, the measuring unit transmits to the image generation apparatus a timing signal for displaying the measurement result in the display section of the image generation apparatus.

12. The measurement apparatus according to claim 1, wherein
the measuring unit further includes a display section for displaying a measurement result.

13. The measurement apparatus according to claim 1, wherein
the touching unit supplies the ultrasonic signal to a liver, and
the measuring unit measures a content percentage of fat in the liver.

14. The measurement apparatus according to claim 1, wherein the response signal is a reflected signal that has been reflected from the inner portion of the body part.

15. A measurement method comprising:
switching between connecting a touching unit to a measuring unit and connecting the touching unit to an image generation apparatus;
supplying an ultrasonic signal to a body part;
receiving, with the touching unit, a response signal that has passed through an inner portion of the body part;
when the touching unit is connected to the measuring unit, measuring, with the measuring unit, a characteristic of the body part based on the received response signal; and
when the touching unit is connected to the image generation apparatus, generating, with the image generation apparatus, an image of a region including the body part in response to the touching unit scanning the region.

16. An ultrasonic diagnosis apparatus comprising:
the measurement apparatus according to claim 1; and
an image generation apparatus that is connected to the touching unit and generates an image of a region including the body part, in response to the touching unit scanning the region, wherein
the measuring unit detects content percentage of fat at a designated measurement point in the image of the region, by having the switching section connect the touching unit to the measuring unit.
